SEQUENCE LISTING (11 sequences)

SEQ ID NO: 1 - protein, 15 aa, Artificial Sequence
  mimetic peptide for apolipoprotein B-100 epitope
RNVPPIFNDVYWIAF

SEQ ID NO: 2 - protein, 15 aa, Artificial Sequence
  memetic peptide for apolipoprotein B-100 epitope
RFRGLISLSQVYLDP

SEQ ID NO: 3 - protein, 15 aa, Artificial Sequence
  mimetic peptide for apolipoprotein B-100 epitope
SVCGCPVGHHDVVGL

SEQ ID NO: 4 - DNA, 204 bp, Artificial Sequence
  DNA sequence for terameric mimetic peptide
gtcgaccgtaatgttcctcctatcttcaatgatgtttattggattgcattcctcgaccgtaatgttcctcctatcttcaatgatgtttattggattgcattcctcgaccgtaatgttcctcctatcttcaatgatgtttattggattgcattcctcgaccgtaatgttcctcctatcttcaatgatgtttattggattgcattc

SEQ ID NO: 5 - protein, 68 aa, Artificial Sequence
  amino acid sequence for terameric mimetic peptide
VDRNVPPIFNDVYWIAFLDRNVPPIFNDVYWIAFLDRNVPPIFNDVYWIAFLDRNVPPIFNDVYWIAF

SEQ ID NO: 6 - protein, 61 aa, Artificial Sequence
  Rabbis virus Nucleoprotein
DVALADDGTVNSDDEDYFSGETRSPEAVYTRIMMNGGRLKRSHIRRYVSVSSNRHARPNLD

SEQ ID NO: 7 - protein, 55 aa, Artificial Sequence
  Hepatitis B virus preS2
MQWNSTTFHQALLDPRVAGLYFPAGGSSSGTVNPVPTTASPISSIFSKTGDPAPN

SEQ ID NO: 8 - protein, 31 aa, Artificial Sequence
  Mouse apolipoprotein C-II
KLRDMYSKSSAAMSTYAGIFTDQLLTLLRGE

SEQ ID NO: 9 - protein, 182 aa, Artificial Sequence
  amino acid sequence of B4RCII
MRGSHHHHHHGSDDDDKIVDRNVPPIFNDVYWIAFLDRNVPPIFNDVYWIRNVPPIFNAFLDDVYWIAFLDRNVPPIFNDVYWIAFLTDVALADDGTVNSDDEDYFSGETRSPEAVYTRIMMNGGRLKRSHIRRYVSVSSNRHARPNLDDEKLRDMYSKSSAAMSTYAGIFTDQLLTLLRGE

SEQ ID NO: 10 - protein, 181 aa, Artificial Sequence
  amino acid sequence of B4RB2
MRGSHHHHHHGSDDDDKIVDRNVPPIFNDVYWIAFLDRNVPPIFNDVYWIAFLDRNVPPIFNDVYWIAFLDRNVPPIFNDVYWIAFLTDVALADGTVNSDDEDYFSGETRSPEAVYTRIMMNGGRLKRSHIRRYVSVSSNRHARPNLDLERNVPPFNDVYWIAFLDRNVPPIFNDVYWIAF

SEQ ID NO: 11 - protein, 175 aa, Artificial Sequence
  amino acid sequence of B4TB2
MRGSHHHHHHGSDDDDKIVDRNVPPIFNDVYWIAFLDRNVPPIFNDVYWIAFLDRNVPPIFNDVYWIAFLDRNVPPIFNDVYWIAFLTMQWNSTTFHQALLDPRVAGLYFPAGGSSSGTVNPVPTTASPISSIFSKTGDPAPNLERNVPPIFNDVYWIAFDRVPPIFNDVYWIAF

(19) United States
(12) Patent Application Publication
(10) Pub. No.: US 2011/0002955 A1
(43) Pub. Date: Jan. 6, 2011

(54) ANTI-OBESE IMMUNOGENIC HYBRID POLYPEPTIDES AND ANTI-OBESE VACCINE COMPOSITION COMPRISING THE SAME

(75) Inventors: Hyo-Joon Kim, Gyeonggi-do (KR); Hee-Jong Lee, Gyeonggi-do (KR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: SJ BIOMED INC., Gyeonggi-do (KR)

(21) Appl. No.: 12/442,760

(22) PCT Filed: Sep. 21, 2007
(86) PCT No.: PCT/KR07/04692

§ 371 Date: Mar. 25, 2009

(30) Foreign Application Priority Data
Sep. 25, 2006 (KR) 10-2006-0093130

(51) Int. Cl. A61K 39/29; C07K 19/00; A61K 39/12; C07H 21/00; C12N 15/63; C12N 5/10; C12P 21/02; A61P 3/04

(52) U.S. Cl. 424/189.1; 530/403; 424/186.1; 536/23.4; 435/320.1; 435/325; 435/69.7

(57) ABSTRACT

Disclosed is an immunogenic hybrid polypeptide for the prevention and treatment of obesity, in which a mimetic peptide of a B cell epitope of apolipoprotein B-IOO; a rabies virus helper T cell epitope or hepatitis B virus surface antigen helper T cell epitope and a C-terminal peptide fragment of mouse apolipoprotein Cu or a mimetic peptide of a B cell epitope of apolipoprotein B-100 are fused to each other in that order in the direction from the N terminus to the C terminus thereof. Also, a vaccine composition for the prevention and treatment of obesity, comprising the immunogenic hybrid polypeptide is disclosed, along with a polynucleotide encoding the immunogenic hybrid polypeptide, a recombinant expression vector carrying the polynucleotide, a host cell anchoring the recombinant expression vector, and a method for producing the immunogenic hybrid polypeptide by culturing the host cell transformed with the recombinant expression vector.

### TECHNICAL FIELD

The present invention relates to an immunogenic hybrid polypeptide, in which a mimetic peptide of a B cell epitope of apolipoprotein B-100; a rabies virus helper T cell epitope or hepatitis B virus surface antigen helper T cell epitope and a C-terminal peptide fragment of mouse apolipoprotein CII or a mimetic peptide of a B cell epitope of apolipoprotein B-100 are fused to each other in that order in the direction from the N terminus to the C terminus thereof. Also, the present invention relates to a vaccine composition for the prevention and treatment of obesity, comprising the immunogenic hybrid polypeptide as an active ingredient. Further, the present invention is concerned with a polynucleotide encoding the immunogenic hybrid polypeptide, a recombinant expression vector carrying the polynucleotide, a host cell transformed with the recombinant expression vector, and a method for producing the immunogenic hybrid polypeptide by culturing the host cell transformed with the recombinant expression vector.

### BACKGROUND ART

Recently, diabetes, arteriosclerosis and coronary atherosclerotic disease (CAD) have been gradually increasing in Korea due to a shift to Western dietary habits, and it is applied to pets like dogs or cats, or domestic animals as well as humans. Serum lipids causing these diseases include cholesterol, triglycerides (TG), free fatty acids and phospholipids. These serum lipids form lipoproteins with apolipoproteins and are transported through the bloodstream. Among them, very low density lipoproteins (VLDL) and low density lipoproteins (LDL) function to transport mainly TG and cholesterol, and changes in LDL-cholesterol levels are indications of the prognosis of the diseases. LDL-cholesterol, which is a major factor of lipid metabolism-associated diseases of adult people, binds to LDL receptors on the plasma membrane of cells in each tissue and is stored and used in the tissue. Alternatively, LDL-cholesterol is, taken up by scavenger cells and hydrolyzed, and free cholesterol is transferred to HDL along with apo E lipoprotein to be recycled in the liver, or is converted to bile salt to be discharged. During this process, the apolipoprotein performs very important functions to maintain structural homeostasis of lipoproteins, serves as a cofactor of the enzyme lipoprotein lipase, and plays a critical role in binding to a specific receptor on the plasma membrane.

Apolipoprotein B-100 (Apo B-100) is a major protein component of LDL, and is also present in IDL and VLDL. Thus, when antibodies in the blood are induced to recognize apo B-100, LDL clearance by phagocytes will easily occur. In this regard, some recent studies have been focused on the employment of vaccines to decrease plasma LDL-cholesterol levels and reduce the incidence of arteriosclerosis. Antibodies induced by such anti-cholesterol vaccine therapy are IgM types which are considered to bind to VLDL, IDL and LDL, and such a strategy suggests the possibility of developing vaccines for preventing and treating hypercholesterolemia and atherosclerosis (Bailey, et al., Cholesterol vaccines. Science 264, 1067-1068, 1994; Palinski W et al., Proc Natl Acad Sci U.S.A. 92, 821-5, 1995; Wu R, de Faire U et al., Hypertension. 33, 53-9, 1999). Also, apolipoprotein B-100 is a huge protein molecule, which consists of 4560 amino acid residues, contains signal peptide of 24 amino acid residues and has a molecular weight of more than 500 kDa (Elovson J et al., Biochemistry, 24:1569-1578, 1985). Since apolipoprotein B-100 is secreted mainly by the liver and is an amphipathic molecule, it can interact with the lipid components of plasma lipoproteins and an aqueous environment (Segrest J. P et al., Adv. Protein Chem., 45:303-369, 1994). Apolipoprotein B-100 stabilizes the size and structure of LDL particles and plays a critical xole in controlling the homeostasis of plasma LDL-cholesterol through binding to its receptor (Brown M S et al., Science, 232:34-47, 1986).

Korean Pat. No. 10-0639397, issued to the present inventors, discloses a mimetic peptide for an epitope of apolipoprotein B-100 which shows inhibitory effects on obesity, an immunogenic hybrid polypeptide (B4T) in which the mimetic peptide is fused to a helper T cell epitope, and an anti-obesity composition comprising the same. However, the hybrid polypeptide resulting from the fusion of the mimetic peptide of B cell epitope of apolipoprotein B-100 with the helper T cell epitope is not expected to be equally effective in the prevention and treatment of animals as in humans due to difference in immunity-associated materials and metabolisms therebetween. In addition, although it is immunized within one group, the fused polypeptide elicits immune responses to a large degree of deviation according to individuals because the folding stability thereof is low.

On the other hand, Many attempts to fuse a hapten with a carrier protein were made to enhance the immunogenicity of the hapten, but failed to obtain uniform enhancing effects. In particular, the linear linkage of a B cell epitope and a T cell epitope, like the present invention, resulted in loss of immunogenicity according to the orientation of the epitopes, the type of each epitope, and the like (Francis, M. J. et al., Nature 330:168-170, 1987), and the presence of a linker brought about reduced antigenicity (Partidos, C. et al., MoI. Immunol. 29:651-658, 1992). That is, there is no consistent rule applicable to design peptide vaccines, and the efficacy of designed vaccines is also not predictable. For the same reasons, when a highly hydrophobic mimetic peptide of a B cell epitope of apolipoprotein B-100 fused with a rabies virus helper T cell epitope, hepatitis B virus surface antigen helper T cell epitope or apolipoprotein C—II, an antigenic region can be internalized into the fusion protein, leading to a decrease in its ability to induce antibody responses.

### DISCLOSURE OF INVENTION

### Technical Problem

Leading to the present invention, the present inventors were conducted intensive and thorough research for providing a stable anti-obesity vaccine which is applicable to animals, such as dogs, cattle, etc. as well as humans and can elicit uniform antibody reactions throughout individuals.

Accordingly, the present inventors have surprisingly found out that a hybrid polypeptide comprising atetrameric mimetic peptide for a B-cell epitope of apolipoprotein B-100 (B4), either a rabies virus helper T cell epitope (R) or hepatitis B virus surface antigen helper T cell epitope (T), and either a C-terminal peptide fragment (CII) of apolipoprotein CII or a dimeric mimetic peptide for a B cell epitope of apolipoprotein B-100 (B2) in that order from the N-terminus thereof, can be effectively applied to the prevention or treatment of obesity in animals as well as humans, in addition to showing excellent immunostimulative effects, thereby completing the present invention.

### Technical Solution

Therefore, it is an object of the present invention to provide an immunogenic hybrid polypeptide in which a tetrameric mimetic peptide of a B-cell epitope of apolipoprotein B-100, either a rabies virus helper T cell epitope or hepatitis B virus surface antigen helper T cell epitope, and either a C-terminal peptide fragment of apolipoprotein CII or a dimeric mimetic peptide for a B cell epitope of apolipoprotein B-100 are fused in that order from the N-terminus thereof.

It is another object of the present invention to provide a vaccine composition for preventing or treating obesity, comprising an immunogenic hybrid polypeptide.

It is still another object of the present invention to provide a polynucleotide encoding the immunogenic hybrid polypeptide.

It is still another object of the present invention to provide a recombinant expression vector comprising the polynucleotide.

It is still another object of the present invention to provide a host cell transformed with the recombinant expression vector.

It is still another object of the present invention to provide a method of producing the immunogenic hybrid polypeptide by culturing a host cell transformed with the recombinant expression vector.

### BRIEF DESCRIPTION OF THE DRAWINGS

FIG. 1(a) is a photograph after a PCR product of an apolipoprotein CII gene was electrophoresed in lane 2, along with a 25/100 by mix DNA Ladderin lane 1, on 2% agarose gel in a TBE buffer system, with a load of 2 □ per well.

FIG. 1(b) is a photograph showing the insertion of a polynucleotide fragment of interest into a recombinant ApoCII/pQE30 vector transformed into E. coli JM109.

FIG. 2(a) is a photograph after a PCR product of an RVNP gene was electrophoresed in lane 2, along with a 100 by ladder (Bioneer) in lane 1, on 2% agarose gel in a TBE buffer system, with a load of 2 □per well.

FIG. 2(b) is a photograph showing the insertion of a SalI-digested fragment in an appropriate direction into a recombinant B4RCII/pQE30 vector transformed into E. coli JM109.

FIG. 2(c) is a photograph showing the insertion of a SalI-digested fragment in an appropriate direction into a recombinant B4RB2/pQE30 vector transformed into E. coli JM109.

FIG. 2(d) is a photograph showing the insertion of a SalI/HindIII-digested fragment in an appropriate direction into a recombinant B4TB2/pQE30 vector transformed into E. coli JM109.

FIG. 3 is a schematic diagram illustrating the procedure of preparing a recombinant expression vector for the expression of a B4RCII fusion polypeptide.

FIG. 4 is a schematic diagram illustrating the procedure of preparing a recombinant expression vector for the expression of a B4RB2 fusion polypeptide.

FIG. 5 is a schematic diagram illustrating the procedure of preparing a recombinant expression vector for the expression of a B4TB2 fusion polypeptide.

FIG. 6 shows a nucleotide sequence of pB4RCII along with the amino acid sequence encoded thereby, which was identified by DNA sequencing.

FIG. 7 shows the nucleotide sequence of pB4RB2 along with the amino acid sequence encoded thereby, which was identified by DNA sequencing.

FIG. 8 shows the nucleotide sequence of pB4TB2 along with the amino acid sequence encoded thereby, which was identified by DNA sequencing.

FIG. 9(a) is an SDS-PAGE photograph showing a change in the expression of B4RCII with time, wherein B4RCII obtained from Escherichia coli M15/pB4RCII 1-4 hours after IPTG induction was electrophoresed in lanes 2 to 5, along with a marker (NEB) in lane M and non-IPTG-induced E. coli M15/pB4RCII in lane 1.

FIG. 9(b) is an SDS-PAGE photograph showing a change in the expression of B4RB2 with time, wherein B4RB2 obtained from Escherichia coli M15/pB4RB2 2˜5 hours after IPTG induction was electrophoresed in lanes 2 to 5, along with a marker (NEB) in lane M and non-IPTG-induced E. coli M15/pB4RB2.

FIG. 9(c) is an SDS-PAGE photograph showing a change in the expression of B4TB2 with time, wherein B4TB2 obtained from Escherichia coli M15/pB4TB2 3˜5 hours after IPTG induction was electrophoresed in lanes 2 and 3, along with a marker (NEB) in lane M1, non-IPTG-induced E. coli M15 in lane 1, a total soluble protein in lane 4, and a total protein solubilized by 8M urea in lane 5.

FIG. 9(d) is a photograph showing the presence of B4RCII through Western blotting analysis using a rabbit anti-PB 14 polyclonal antibody.

FIG. 10 shows the elution of B4RCII from resin-bound B4RCII according to linear imidazole concentration gradients in a graph (a) and in an SDS-PAGE photograph (b) (M1: NEB prestained marker, lane 1: no induction cell crude extract, lane 2: 4 hr-induction cell crude extract, lane 3: total soluble protein, lane 4: total protein solubilized by 8M urea (before resin binding), lane 5: flow-through, lane 6: wash fraction (50 mM imidazole), and lane 7: eluted fraction (500 mM imidazole), a load of 7.5 □/well).

FIG. 10 shows the elution of B4RB2 from resin-bound B4RB2 according to linear imidazole concentration gradients in a graph (c) and in an SDS-PAGE photograph (d) (lane 1: Elpis prestained protein marker, lane 2: total soluble protein, lane 3: total protein solubilized by 8M urea (before resin biding), lane 4: flow-through, lane 6: eluted fraction (500 mM imidazole), a load of 3 □/well).

FIG. 10 shows the elution of B4TB2 from resin-bound B4TB2 according to linear imidazole concentration gradients in a graph (e) and in an SDS-PAGE photograph (f) (lane 1: Elpis prestained protein marker, lane 2: total soluble protein, lane 3: total protein solubilized by 8M urea (before resin binding), lane 4: flow-through, lane 5: wash fraction (50 mM imidazole), lane 6: eluted fraction (500 mM imidazole), lane 7: eluted fraction (500 mM imidazole), a load of 7.5 □/well).

FIG. 11 is a graph showing weight gains of C57BL/6 mouse groups which were immunized with B4RCII, B4RB2 and B4TB2 at time points indicated by red arrows, with a DIO starting point indicated by a blue arrow.

FIG. 12 is a graph showing changes in the titer of anti-B4 antibody over time for animals immunized with B4RCII, B4RB2 and B4TB2.

FIG. 13 is a graph showing blood lipid levels of animals one week after tertiary boosting with the vaccines of the present invention (16-week-old).

### BEST MODE FOR CARRYING OUT THE INVENTION

In accordance with an aspect thereof, the present invention is directed to an immunogenic hybrid polypeptide in which a mimetic peptide of a B-cell epitope of apolipoprotein B-100, either a rabies virus helper T cell epitope or a hepatitis B virus surface antigen helper T cell epitope, and either a C-terminal peptide fragment of apolipoprotein CII or a dimeric mimetic peptide for a B cell epitope of apolipoprotein B-100 are fused, in that order, from the N-terminus thereof.

The term “mimetic peptide of an epitope”, as used herein refers to a peptide that mimics a minimal part of the epitope, which is an epitope that is sufficiently similar to a native epitope so that it can be recognized by an antibody specific to the native epitope, or that is able to increase an antibody to crosslink with a native epitope. A mimetic peptide is also called a mimotope. Such a mimetic peptide is advantageous because it is recognized as “non-self” in vivo and thus overcomes the problem of self-tolerance in immune responses. The mimetic peptide of a B cell epitope of apo B-100 is recognized by an antibody specifically binding to apo B-100. The antibody specifically binding to apo B-100 includes polyclonal and monoclonal antibodies, which specifically recognize and bind to apo B-100, and fragments thereof, for example, Fc, Fab and F(ab′)₂. Among them, monoclonal antibodies is preferred, Mab B9 and Mab B23 are more preferred.

The mimetic peptide of a B cell epitope of apo B-100 according to the present invention includes an amino acid sequence selected from the group consisting of SEQ ID No.: 1, SEQ ID No.: 2 and SEQ ID No.: 3. The present inventors isolated mimetic peptides (SEQ ID Nos.1, 2 and 3) that are recognizable by a monoclonal antibody against apo B-100, Mab B9 or Mab B23, from a phage displayed peptide library by biopanning with the library. The mimetic peptide of the epitope of apo B-100, which includes an amino acid sequence selected from the group consisting of SEQ ID No.: 1, SEQ ID No.: 2 and SEQ ID No.: 3., may be in a monomeric form that is composed of a single copy of the amino acid sequence having any one of the SEQ ID Nos., or, to further enhance the immunogenicity of the mimetic peptide, may be in a multimeric form in which two or more, preferably three to eight, and more preferably three to six copies of the amino acid sequence having any one of the SEQ ID Nos. are linked. Most preferred is a tetramer in which four copies are linked. When the mimetic peptide is in a multimeric form, amino acid sequences each of which constitutes a monomer may be covalently linked directly or via a linker. When the amino acid sequences are linked via a linker, the linker may consist of one to five amino acid residues, which are selected from, for example, glycine, alanine, valine, leucine, isoleucine, proline, serine, threonine, asparagine, aspartic acid, cysteine, glutamine, glutamic acid, lysine and arginine. Preferred amino acids available in the linker may include valine, leucine, aspartic acid, glycine, alanine and proline. More preferably, taking the ease of gene manipulation into account, two amino acids selected from valine, leucine, aspartic acid, etc. may be linked and used as a linker. A preferred mimetic peptide is prepared by linking two or more copies of an amino acid sequence selected from SEQ ID Nos. 1, 2 and 3 via the linker.

The term “T cell epitope”, as used herein, refers to an amino acid sequence that is able to bind to MHC ClassII molecules with a suitable efficiency and stimulate T cells or bind to T cells in a complex with MHC ClassII In this case, the T cell epitope is recognized by a specific receptor present on T cells, and functions to provide a signal requiring the differentiation of B cells to antibody-producing cells and induce cytotoxic T lymphocytes (CTL) to destroy target cells. For the purpose of the present invention, helper T cell epitopes are preferably used as recognition targets of the specific receptor. Of them, a rabies virus helper T cell epitope or a hepatitis B virus surface antigen helper T cell epitope is found to elicit better effects.

The rabies virus infects into livestock and wild animals as well as pets, such as dogs and cats, causing acute encephalitis. Rabies can be prevented by vaccination, both in humans and other animals. For use in the present invention, a peptide fragment (R), 58 amino acids long, containing a helper T cell epitope of a host, was prepared from a rabies virus ribonucleoprotein gene (NCBI gene ID; AF406695) through gene manipulation, the amino acid sequence of which is represented by SEQ ID NO. 6 (Ertl, H. C. J., et al., Journal of Virology, 63(7), 2885-2892, 1989).

The genome of hepatitis B virus (HBV) is 3.2 kb in length, possesses the information for four important proteins and contains four open reading frames, S gene (surface antigen protein), C gene (core protein), P gene (DNA polymerase) and X gene. The S gene is divided into an S region encoding HBsAg and a preS region. The preS region is divided into preS1 encoding 108 or 119 amino acids according to HBV strains and preS2 encoding 55 amino acids regardless of subtype. The HBV preS2 protein activates helper T cells during in vivo immune responses, thereby stimulating the formation of an antibody against HBV. SEQ ID No. 7 indicates a amino acid sequence of HBV helper T cell epitope.

At the C terminal region of the immunogenic hybrid polypeptide is located a C-terminal peptide fragment of mouse apolipoprotein CII or a mimetic peptide of a B cell epitope of apolipoprotein B-100.

Mouse apolipoprotein CII, consisting of 79 amino acid residues with a molecular weight of 8,800 Da (Hoffer, M. J., et al., Genomics 17(1), 45-51, 1993), is produced mainly in the small intestine and the liver, and can be found in chylomicron, VLDL and HDL, functioning as an essential cofactor for the enzymatic activity of apolipoprotein lipase (LPL) (Storjohann, R., et al., Biochimica et Biophysica Acta, 1486, p 253-264, 2000). In a preferable example of the present invention, a peptide consisting of the 33 C-terminal amino acid residues of apolipoprotein CII, which are responsible for the control of LPL activity, was cloned from a mouse apolipoprotein CII gene (NCBI gene ID; NM009695) and is represented by SEQ ID NO.: 8.

The mimetic peptide of an epitope of apolipoprotein B-100, which is provided to the C-terminal region of the immunogenic hybrid polypeptide of the present invention, contains an amino acid sequence selected from the group consisting of SEQ ID NO.: 1, SEQ ID NO.: 2 and SEQ ID NO.: 3. The mimetic peptide of an epitope of apo B-100, which includes an amino acid sequence selected from the group consisting of SEQ ID NO.: 1, SEQ ID NO.: 2 and SEQ ID NO.: 3., may be in a monomeric form that is composed of a single copy thereof. In order to further enhance the immunogenicity thereof, the mimetic peptide may be in a multimeric form, in which two to four copies of the amino acid sequence are linked, with greater preference for a dimeric form, consisting of two copies. In the case of a multimeric form, amino acid sequences, each of which constitutes a monomer, may be covalently linked directly or via a linker.

The term “immunogenicity”, as used herein, refers to the ability to induce both cellular and humoral immune responses to defend the body against impurities. A material inducing such immune responses is called an immunogen. In the fusion polypeptide according to the present invention, a B cell epitope of apolipoprotein B-100, a rabies virus helper T cell epitope or a hepatitis B virus surface antigen helper T cell epitope, and a C-terminal peptide fragment of apolipoprotein CII are employed as immunogens.

When a B cell epitope and a T cell epitope are fused to form an immunogenic polypeptide, like the polypeptide of the present invention, it is known that the B cell epitope should be exposed outside the folding structure of the polypeptide with the T cell epitope located internally, in order to induce effective immune responses (Partidos C, et al., Eur J. Immunol., 22(10):2675-80, 1992). In addition to the architecture of the prior art B4T fusion protein, in which only a mimetic peptide of a B cell epitope of apolipoprotein B-100 and a T cell epitope are linked, a fragment of apolipoprotein CII or a mimetic peptide of a B cell epitope of apolipoprotein B-100 is linked to the C-terminus of the T cell epitope in accordance with the present invention. The resulting fusion polypeptide is found to be improved in the stability of the protein folding structure and to induce uniform antibody reactions throughout individuals as the T cell epitope is further surrounded by the B cell epitope, with minimal exposure to the outside.

The term “polypeptide”, as used herein, is a term including a full-length amino acid chain in which residues including two or more amino acids are conjugated by covalent peptide bonds, and includes dipeptides, tripeptides, oligopeptides and polypeptides. In particular, in the present invention, the polypeptide means a hybrid polypeptide in which two or more peptides, in which several to several tens of amino acids are covalently bonded, are linked with each other. Each peptide sequence comprising the polypeptide includes a sequence corresponding to the aforementioned epitope, and may further include a sequence adjacent to the epitope. These peptides may be made of L- or D-amino acids, or may be in various combinations of amino acids in two different configurations.

The term “hybrid polypeptide”, as used herein, generally indicates a peptide in which heterogeneous peptides having different origins are linked. In the present invention, hybrid polypeptide is a peptide in which a B-cell epitope, either a rabies virus helper T cell epitope or a hepatitis B virus surface antigen helper T cell epitope, and either a C-terminal peptide fragment of apolipoprotein CII or a mimetic peptide of a B cell epitope of apolipoprotein B-100 are arranged in that order from the N terminus to the C terminus, with a linkage therebetween.

In a preferred embodiment according to the present invention, the hybrid polypeptide is a polypeptide (B4RCII) in which four copies of the amino acid sequence of SEQ ID NO.: 1 (B4), a rabies virus helper T cell epitope (R), and a C-terminal peptide fragment (CII) of mouse apolipoprotein CII are linked sequentially from the N terminus to the C terminus (SEQ ID NO.:9). In another preferred embodiment of the present invention, the hybrid polypeptide is a polypeptide (B4RB2) which comprises four copies of the amino acid sequence of SEQ ID NO.: 1 (B4), a rabies virus helper T cell epitope (R), and two copies of the amino acid sequence of SEQ ID NO.: 1 (B2), linked sequentially from the N terminus to the C terminus (SEQ ID NO: 10). In a still another preferred embodiment of the present invention, the hybrid polypeptide is a polypeptide (B4TB2) which comprises four copies of the amino acid sequence of SEQ ID NO.: 1 (B4), a hepatitis B virus surface antigen helper T cell epitope (T), and two copies of the amino acid sequence of SEQ ID NO.: 1 (B2), linked sequentially from the N terminus to the C terminus (SEQ ID NO: 11).

In accordance with the present invention, the hybrid polypeptide may consist completely of immunogenic portions including a B cell epitope, a rabies virus helper T cell epitope or a hepatitis B virus surface antigen helper T cell epitope, a C-terminal peptide fragment of apolipoprotein CII, and an adjacent sequence thereof, and may optionally further comprise an additional sequence. However, the additional sequence is preferably configured to prevent a reduction in overall immunogenicity. The additional sequence includes a linker sequence. In the case where linkers are used to link the epitopic regions therethrough, they must be selected in order not to negatively affect the induction of immune responses.

In another aspect, the present invention relates to a recombinant vector comprising a polynucleotide encoding the immunogenic hybrid polypeptide, and a recombinant expression vector comprising the polynucleotide and, and a host cell transformed with the recombinant expression vector, and a method of producing the immunogenic hybrid polypeptide by culturing a host cell transformed with the recombinant expression vector.

The immunogenic hybrid polypeptide of the present invention may be produced by chemical synthesis or genetic recombination. In detail, a process of producing the immunogenic hybrid polypeptide of the present invention by genetic recombination comprises the following four steps:

The first step is to insert a gene encoding the hybrid polypeptide into a vector to construct a recombinant vector. A vector into which foreign DNA is introduced may be a plasmid, a virus, a cosmid, or the like. The recombinant vector includes a cloning vector and an expression vector. A cloning vector contains a replication origin, for example, a replication origin of a plasmid, pharge or cosmid, which is a “replicon” at which the replication of an exogenous DNA fragment attached thereto is initiated. An expression vector was developed for use in protein synthesis. A recombinant vector serves as a carrier for a foreign DNA fragment inserted thereto, which typically means a double-stranded DNA fragment. The term “foreign DNA”, as used herein, refers to DNA derived from a heterogeneous species, or a substantially modified form of native DNA from a homogenous species. Also, the foreign DNA includes a non-modified DNA sequence that is not expressed in cells under normal conditions. In this case, a foreign gene is a specific target nucleic acid to be transcribed, which encodes a polypeptide. The recombinant vector contains a target gene that is operably linked to transcription and translation expression regulatory sequences, which exert their functions in a selected host cell, in order to increase expression levels of the transfected gene in the host cell. The recombinant vector is a genetic construct that contains essential regulatory elements to which a gene insert is operably linked to be expressed in cells of an individual. Such a genetic construct is prepared using a standard recombinant DNA technique. The type of the recombinant vector is not specifically limited as long as the vector expresses a target gene in a variety of host cells including prokaryotes and eukaryotes and functions to produce a target protein. However, preferred is a vector which is capable of mass-producing a foreign protein in a form similar to a native form while possessing a strong promoter to achieve strong expression of the target protein. The recombinant vector preferably contains at least a promoter, a start codon, a gene encoding a target protein, a stop codon and a terminator. The recombinant vector may further suitably contain DNA coding a signal peptide, an enhancer sequence, 5′- and 3′-untranslational regions of a target gene, a selection marker region, a replication unit, or the like.

The second step is to transform a host cell with the recombinant vector and culture the host cell. The recombinant vector is introduced into a host cell to generate a transformant by a method described by Sambrook, J. et al., Molecular Cloning, A Laboratory Manual (2nd Ed.), Cold Spring Harbor Laboratory, 1.74, 1989, the method including a calcium phosphate or calcium chloride/rubidium chloride method, electroporation, electroinjection, chemical treatments such as PEG treatment, and gene gun. A useful protein can be produced and isolated on large scale by culturing a transformant expressing the recombinant vector in a nutrient medium. Common media and culture conditions may be suitably selected according to host cells. Culture conditions, including temperature, pH of a medium and culture time, should be maintained suitable for cell growth and mass production of a protein of interest. Host cells capable of being transformed with the recombinant vector according to the present invention include both prokaryotes and eukaryotes. Host cells having high introductionefficiency of DNA and having high expression levels of an introduced DNA may be typically used. Examples of host cells include known prokaryotic and eukaryotic cells such as Escherichia sp., Pseudomonas sp., Bacillus sp., Streptomyces sp., fungi and yeast, insect cells such as Spodoptera frugiperda (Sf9), and animal cells such as CHO, COS 1, COS 7, BSC 1, BSC 40 and BMT 10. E. coli may be preferably used.

The third step is to induce the hybrid polypeptide to express and accumulate. In the present invention, the inducer IPTG was used for the induction of peptide expression, and induction time was adjusted to obtain maximal protein yield.

The final step is to isolate and purify the hybrid polypeptide. Typically, a recombinantly produced peptide can be recovered from a medium or a cell lysate. When the peptide is in a membrane-bound form, it may be liberated from the membrane using a suitable surfactant solution (e.g., Triton-X 100) or by enzymatic cleavage. Cells used in the expression of the hybrid peptide may be destroyed by a variety of physical or chemical means, such as repeated freezing and thawing, sonication, mechanical disruption or a cell disrupting agent, and the hybrid peptide may be isolated and purified by commonly used biochemical isolation techniques (Sambrook et al., Molecular Cloning: A laboratory Manual, 2nd Ed., Cold Spring Harbor Laboratory Press, 1989; Deuscher, M., Guide to Protein Purification Methods Enzymology, Vol. 182. Academic Press. Inc., San Diego, Calif., 1990). Non-limiting examples of the biochemical isolation techniques include electrophoresis, centrifugation, gel filtration, precipitation, dialysis, chromatography (ion-exchange chromatography, affinity chromatography, immunosorbent affinity chromatography, reverse phased HPLC, gel permeation HPLC), isoelectric focusing, and variations and combinations thereof.

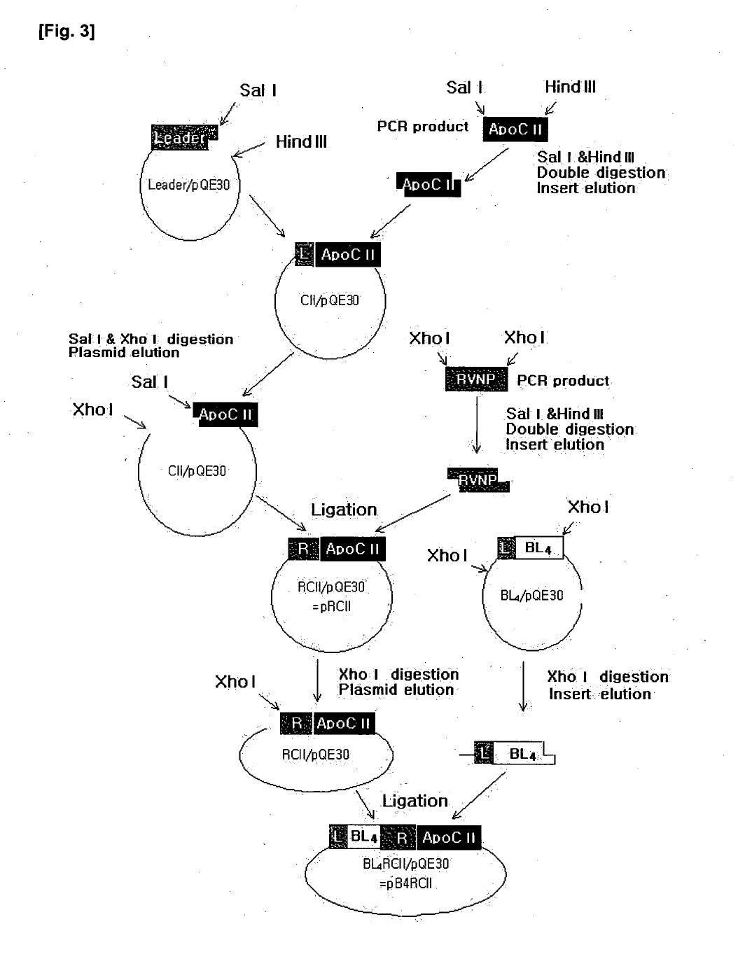

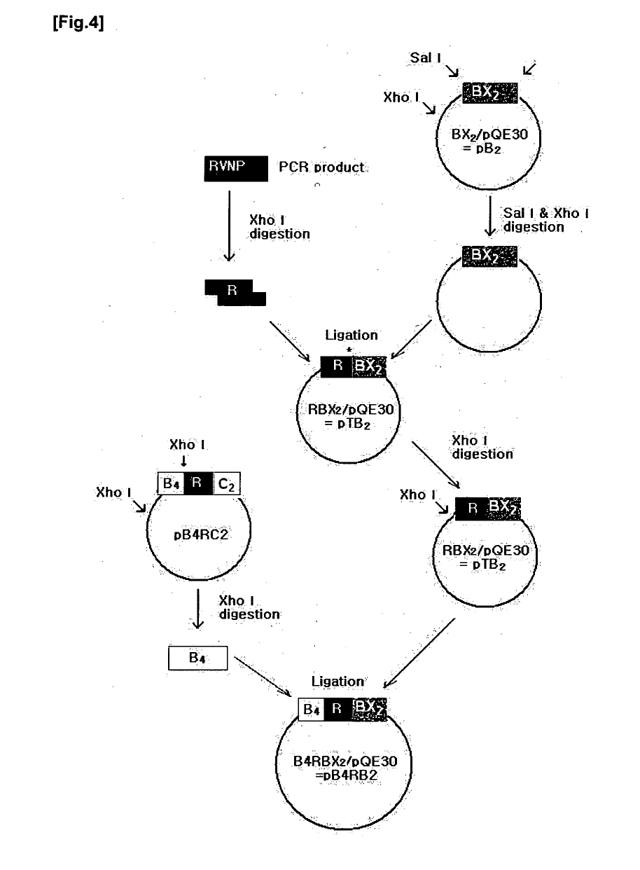

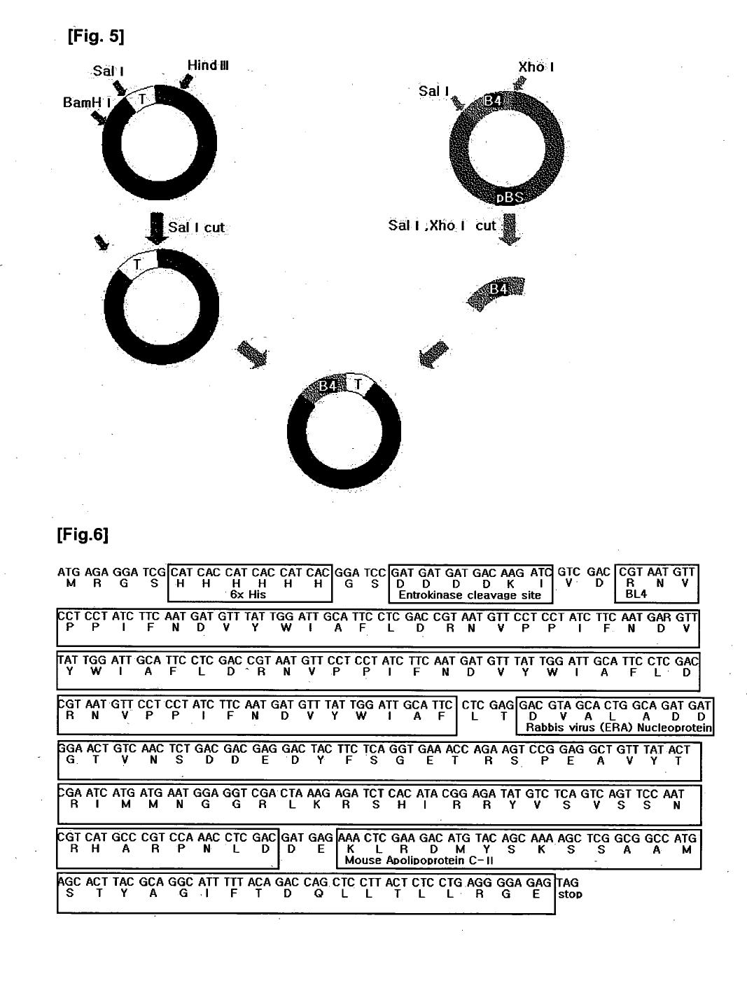

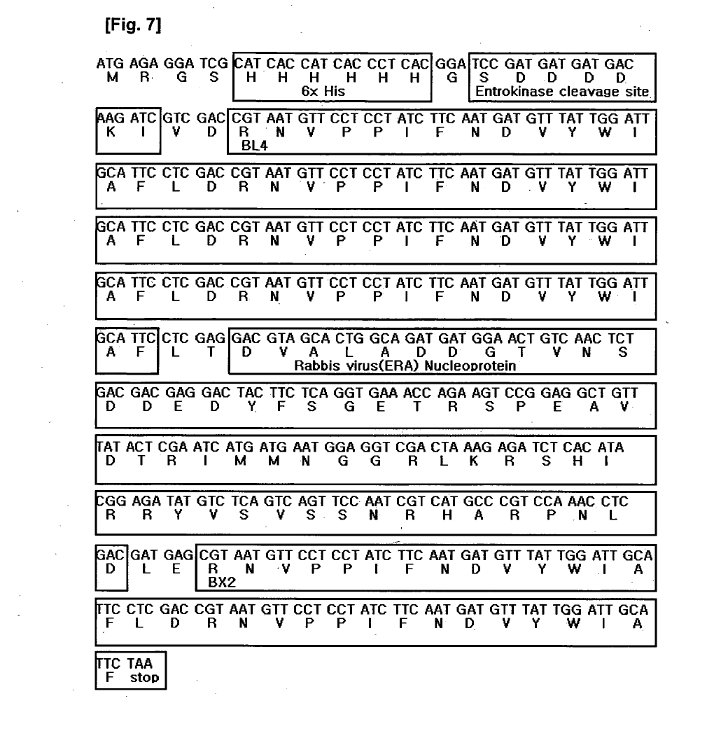

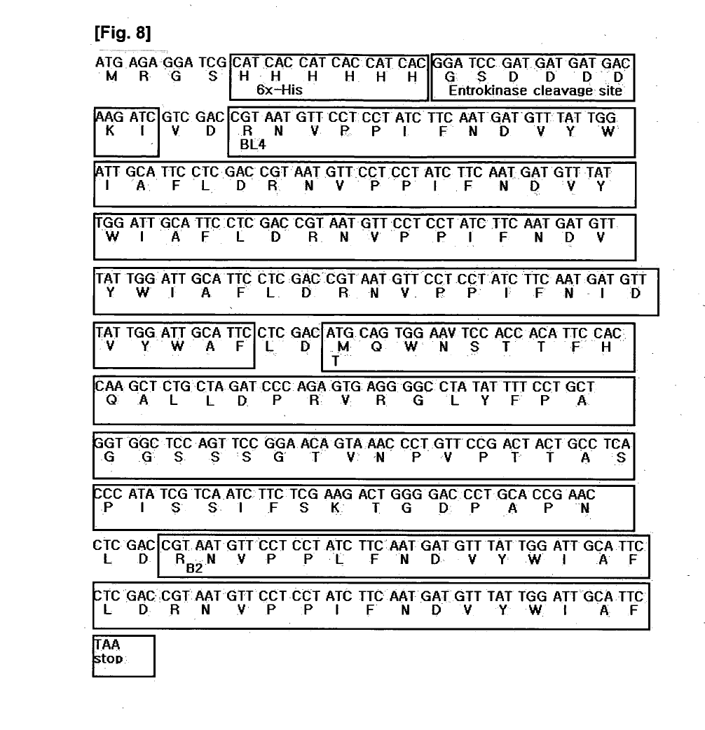

In a preferable embodiment of the present invention, a gene encoding a c-terminal region of B4, a tetrameric form of the mimetic peptide of apolipoprotein B-100 that exhibits anti-obesity activity, a functional peptide containing a B cell epitope but no T cell epitopes, was linked with a part of the gene encoding the rabies viral nucleoprotein containing a T cell epitope (R fragment) and then with a part of the mouse apolipoprotein gene (CII fragment) to construct a B4RCII gene (FIG. 3).

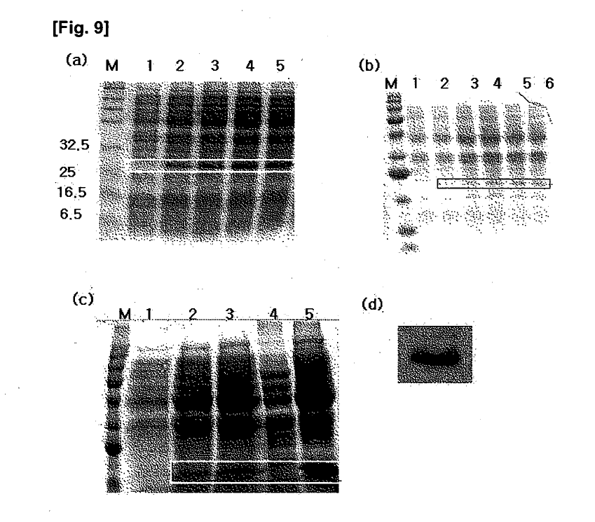

Used in the present invention is the B4 fragment, which was disclosed in Korean Pat. No. 10-0639397. Apolipoprotein CII and RVNP (a Rabies Virus nucleoprotein containing a helper T cell epitope) genes were obtained using RT-PCR.pQE30 was chosen as an expression vector for B4RCII because it initiates protein expression from its internal start codon along with six histidine residues for the convenience of protein purification, followed by anenterokinase cleavage site. The protein thus expressed was found to be approximately 21 KDa in size, as calculated on the basis of the molecular weights of the amino acids thereof, and to be approximately 22 KDa in size, as measured by SDS-PAGE. SDS-PAGE, with samples taken according to times, demonstrates the expression of the protein of interest (FIG. 9).

In another aspect, the present invention relates to a vaccine composition for preventing or treating obesity, comprising an immunogenic hybrid polypeptide.

There is no consistent rule applicable to peptide vaccine design, and the efficacy of designed vaccines is also unpredictable. For the same reasons, when a highly hydrophobic PB14 peptide is fused with a T cell epitope that is a heterogeneous peptide, an antigenic region can be internalized into the fusion protein, leading to a decrease in its ability to induce antibody responses. With this background, in which the result is difficult to interpret, a hybrid polypeptide, in which a mimetic peptide of a B cell epitope of apolipoprotein B-100, a rabies virus helper T cell epitope or a hepatitis B virus surface antigen helper T cell epitope, and a C-terminal peptide fragment of apolipoprotein CII or a mimetic peptide of a B cell epitope of apolipoprotein B-100 were fused in that order in the direction from the N terminus to the C terminus, was constructed and demonstrated to have immunogenicity for anti-obesity.

Rats were immunized with the immunogenic hybrid polypeptide of the present invention expressed and purified by genetic recombination, and the effect of an antigen on the induction of immune responses was assessed by investigating (a) body weight gain, (b) serum antibody titers and (c) changes in serum lipid profiles, thereby determining a highly efficient form of the antigen. As a result, compared to a control group, a group vaccinated with the hybrid polypeptide (B4RCII, B4RB2 and B4TB2) showed suppressed weight gain, high titers and extended retention of an antibody against the mimetic peptide, and decreased serum levels of TG and LDL-cholesterol.

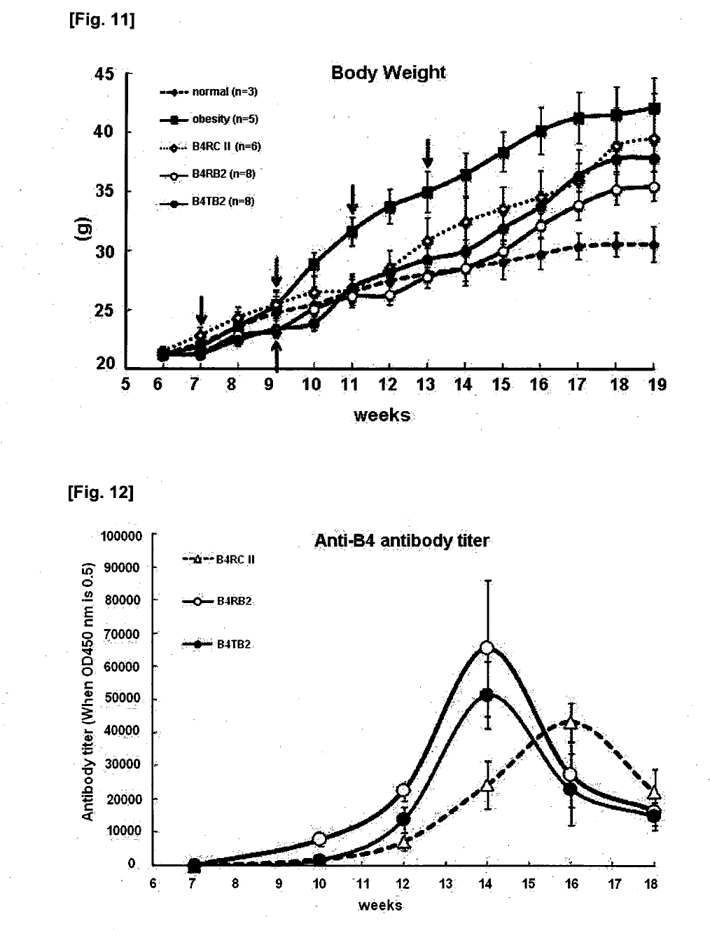

In detail, 50□/150□ of each of purified B4RCII, B4RB2 and B4TB2 were intraperitoneally injected into 6-week-old ICR rats three times at 2-week intervals, and changes in body weight of the rats were observed and plotted on a graph (FIG. 12). After the primary boost, a high-fat diet was provided to the rats in order to cause DIO (diet induced obesity). The individual rats were similar in body weight, ranging from 22 to 23 g, throughout the groups until the primary injection and boost. However, from the start of DIO, the control (obesity) was found to increase in body weight whereas the groups injected with B4RCII, B4RB2 or B4TB2 showed only a slight increase in body weight. When they were 14-weeks-old (8 weeks after the primary injection), the control and the B4RB2-injected group differed in body weight by approximately 8 g, indicating that the weak immune response induced by the primary injection was boosted by the secondary injection to an extent sufficient to suppress weight gain. After the tertiary injection, the weight gain was measured to remain within the expected deviation range.

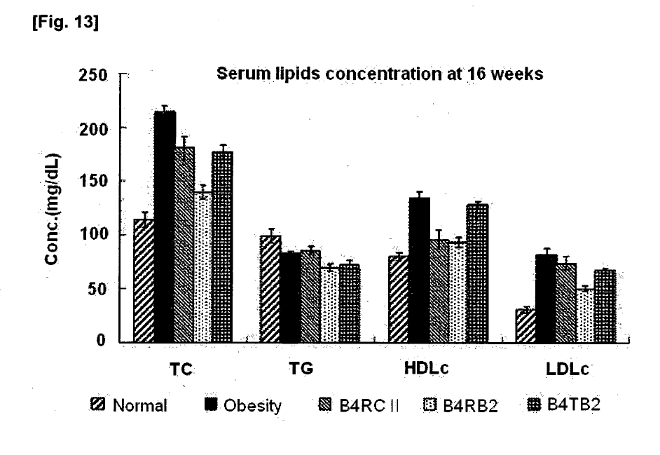

In addition, the ICR mice in the vaccinated groups were analyzed for antibody titer at Week 7, 10, 12, 14, 16 and 18-weeks-old using indirect ELISA (FIG. 12). As for the lipid levels in the blood, the vaccinated groups were found to be lower in total blood cholesterol (TC), triglycerides (TG), HDL cholesterol and LDL cholesterol lipid levels than was the control (FIG. 13).

Taken together, these results demonstrate that the hybrid polypeptides, B4RCII, B4RB2 and B4TB2, according to the present invention can be used as effective anti-obesity vaccines. With the ability to induce more uniform and stable immune responses compared to the conventional hybrid polypeptide B4T, the hybrid polypeptides according to the present invention can be used in the preparation of effective anti-obesity vaccine compositions.

The anti-obesity vaccine of the present invention is composed of an antigen, a pharmaceutically acceptable carrier, a suitable adjuvant and other common materials, and is administered in an immunologically effective amount. The term “immunologically effective amount”, as used herein, refers to an amount that is sufficient to exert the therapeutic and preventive effect on obesity and does not cause side effects or severe or excess immune responses. An accurate dosage may vary according to the specific immunogen to be administered, and may be determined by those skilled in the art using a known method for assaying the development of an immune response. Also, the dosage may vary depending on administration forms and routes, the recipient's age, health state and weight, properties and degree of symptoms, types of currently received therapy, and treatment frequency. The carriers are known in the art and include a stabilizer, a diluent and a buffer. Suitable stabilizers include carbohydrates, such as sorbitol, lactose, mannitol, starch, sucrose, dextran and glucose, and proteins, such as albumin or casein. Suitable diluents include saline, Hanks' Balanced Salts and Ringer's solution. Suitable buffers include an alkali metal phosphate, an alkali metal carbonate and an alkali earth metal carbonate. The vaccine may also contain one or more adjuvants to enhance or strengthen immune responses. Suitable adjuvants include peptides; aluminum hydroxide; aluminum phosphate; aluminum oxide; and a composition that consists of a mineral oil, such as Marcol 52, or a vegetable oil and one or more emulsifying agents, or surface active substances such as lysolecithin, polycations and polyanions. The vaccine composition of the present invention may be administered as an individual therapeutic agent or in combination with another therapeutic agent, and may be co-administered either sequentially or simultaneously with a conventional therapeutic agent. The vaccine composition may be administered via known administration routes. Administration methods include, but are not limited to, oral, intradermal, intramuscular, intraperitoneal, intravenous, subcutaneous, and intranasal routes. Also, a pharmaceutical composition may be administered using a certain apparatus, which can deliver an active material to target cells.

### MODE FOR THE INVENTION

A better understanding of the present invention be obtained through the following examples which are set forth to illustrate, but are not to be construed as the limit of the present invention.

### Example 1

### Preparation of Experimental Materials and Experimental Animals

A DNA miniprep kit and a kit used to extract DNA from a gel were purchased from Nucleogen, Bacto trypton, Bacto yeast extract, agar, etc. from Difco (Detroti, MI), restriction enzymes from Takara, and T4 DNA ligasefrom NEB. pBluescript II SK (Stratagene), PCR 2.1 (Invitrogen, Carlsbad, Calif.) and pQE30 (Qiagen) vectors and E. coli JM109 and M15 strains (Qiagen) were used. IPTG used to induce protein production was purchased from Sigma, the Ni-NTA resin used to purify expressed proteins from Novagen, and the prestained marker used in SDS-PAGE, Western blotting, ECL, etc. from NEB. Urea used to denature proteins was purchased from Duchefa, and immidazole used in protein purification from USB. The membrane used in dialysis was MWCO 3,500 purchased from Spectrum, and the reagent used to prevent protein aggregation was CHAPS from Amresco. The antibody used in ELISA was HRP-conjugated anti-rat IgG from Sigma. The substrate solution used in Western blotting and ECL was BCIP/NBT from Sigma, and the ECL Plus Western Blotting Detection Reagent was purchased from Amersham. Adjuvants used were Freund's adjuvant (Sigma) and aluminum hydroxide (Reheis). Protein concentration was determined by Pierce's BCA protein assay and Biorad's Bradford assay.

6-week-old female ICR mice were purchased from Central Lab.Animal Inc., Korea. ICR mice were bred with a normal diet (Samtako, Inc., natural proteins 18% or higher, crude fats 5.3%, crude fibers 4.5%, minerals 8.0%) until the boost of the immune response, and then with a high-fat diet (60% kCal fat, D12492, Research Diets Inc., New Brunswick, N.J.).

### Example 2

### Mouse ApoCII Gene Cloning

2-1. Isolation of Total RNA from Murine Hepatic Tissue

The isolation of total RNA was performed with TRIzol (Invitrogen). All of the solutions used for RNA isolation were treated with 0.1% diethyl pyrocarbonate-treated water (DEPC-dH₂O) to inhibit RNase activity. 50 □ of hepatic tissues from mice was mixed with 2 □ of TRIzol, followed by homogenization. The homogenate was placed on ice for 20 min and centrifuged at 4° C. at 14,000 rpm for 15 min. The supernatant was transferred to a new tube, with care taken to exclude any protein from the tube. 200 □ of chloroform (Merck) was added to the tube, which was then vortexed for 30 sec. Again, reaction on ice for an additional 20 min was followed by centrifugation t 4° C. at 14,000 rpm for 15 min. Only the supernatant was transferred to a new tube, and mixed with the same volume of phenol/chloroform and 0.2 M sodium acetate (pH 5.2) before vortexing for 5 sec. After being placed on ice for 20 min, the mixture was centrifuged at 4° C. and 14,000 rpm for 15 min. The supernatant was mixed with an equal volume of isopropanol (Merck) and stored at −70° C. for 1 hour, followed by centrifugation at 4° C. and 14,000 rpm for 10 min to form an RNA pellet. This was washed with 1 ml of 75% ethanol, dried and suspended in DEPC-dH₂0 before storage at −70° C. The RNA thus obtained was identified by electrophoresis on 1% agarose gel (0.5% TAE) while RNA concentration was determined using GeneQuant II (Pharmacia biotech).

2-2. cDNA Synthesis from Total RNA

cDNA synthesis was achieved using a cDNA cycle™ kit (Invitrogen). 400 ng of the RNA was placed into a PCR tube and mixed with DEPC-dH₂O to form a final volume of 11.5 □. It was mixed well with 1 □ of oligo-dT primers and reacted for 10 min in a 65° C. water bath and then for 2 min at room temperature. A mixture of RNase inhibitor 1.0 □, 5× RT buffer 4.0 □, 100 mM dNTPs 1.0 □, 80 mM sodium pyrophosphate 1.0 □ and AMV reverse transcriptase 0.5 □ was added to the tube, which was then tapped slightly, placed for 1 hour in a 42° C. water bath and for 2 min at 95° C. and immediately stored on ice. After the addition of 1.0 □ of 0.5 M EDTA (pH 8.0) and 20 □ of phenol-chloroform, the mixture was vortexed and centrifuged at 4° C. at 14,000 rpm for 15 min. The supernatant thus formed was transferred to a new tube, added with 22 □ of ammonium acetate and 88 □ of 75% ethanol, mixed by vortexing, and stored overnight at −70° C. Following centrifugation at 4° C. at 14,000 rpm for 15 min, the resulting pellet was resuspended in 20 □ of deionized water. cDNA was identified by electrophoresis on 1% agarose gel (0.5% TAE buffer).

2-3. PCR of C-Terminal Fragment of Mouse Apolipoprotein CII

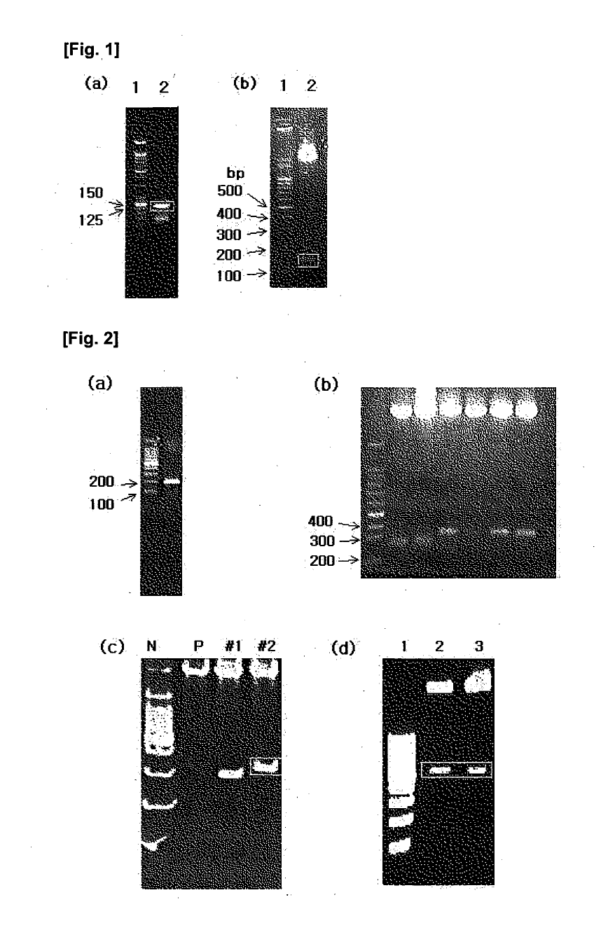

DNA Thermal cycler 480 was used for all PCR in this example. For use in PCR to amplify 99 genes including the lipase activating region of mouse apolipoprotein CII, a set of an apoCII-sense primer (5′-tc aga GTC GAC gat gag aaa ctc agg gac-3′) and an apoCII-antisense primer (5′-tat AAG CTT ggg ctt gcc tgg cag cag cta c-3′) was synthesized. First, to a PCR tube was added 1 □ of each of the apoCII-sense primer and the apoCII-antisense primer (2 pmol/□) and 2 □ of the cDNA synthesized at Example 2-2. Finally, 50 □ of a PCR solution containing 5 □ of 10× buffer, 8 □ of dNTP and 1 □ of Taq DNA polymerase (Takara) was prepared. PCR was started by pre-denaturation at 94° C. for 5 min and then with 30 cycles of denaturation at 98° C. for 30 sec, annealing at 56° C. for 30 sec and extension at 72° C. for 30 sec, followed by extension at 72° C. for 5 min. The PCR product was identified by electrophoresis on 1.5% agarose gel (0.5% TAE buffer) (FIG. 1b).

2-4. Construction of ApoCII/pQE30

The apoCII PCR product was digested with SalI and HindIII. The same restriction enzymes were applied to pQE30.

The CII digest was ligated overnight with the linear pQE30 vector in the presence of T4 DNA ligase at 16° C. pQE30, an expression vector, is designed to produce a protein tagged with 6× histidine, which allows convenient protein purification. The recombinant plasmid thus obtained was transformed into JM109 E. coli and amplified. After preparation from the transformant, the plasmid was treated with SalI and HindIII to identify the insertion of the gene of interest therein.

### Example 3

### Construction of Artificial RCII Gene

3-1. Isolation of Genomic DNA from Rabies Virus) Strain ERA

The isolation of total RNA was performed with TRIzol (Invitrogen). All of the solutions used for RNA isolation were treated with 0.1% diethyl pyrocarbonate-treated water (DEPC-dH₂O) to inhibit RNase activity. For use in the isolation of total RNA, the Rabies virus was obtained from a rabies virus vaccine. First, 200 □ of a 20% (w/v) polyethylene glycol-800 solution containing 2.5 M NaCl was added to 1.2 □ of the vaccine and placed on ice for 1 hour, followed by centrifugation at 4° C. and 14,000 rpm for 10 min. This virus pellet thus obtained was mixed with 1 □ of TRIzol and pipetted sufficiently. The homogenate was placed on ice for 20 min and centrifuged at 4° C. and 14,000 rpm for 15 min. The supernatant was transferred to a new tube, with care taken to exclude any protein from the tube. 200 □ of chloroform (Merck) was added to the tube, which was then vortexed for 30 sec. Again, reaction on ice for an additional 20 min was followed by centrifugation at 4° C. and 14,000 rpm for 15 min. The supernatant alone was transferred to a new tube and mixed with the same volume of phenol/chloroform and 0.2 M sodium acetate (pH 5.2) before vortexing for 5 sec. After being placed on ice for 20 min, the mixture was centrifuged at 4° C. and 14,000 rpm for 15 min. The supernatant was mixed with an equal volume of isopropanol (Merck) and stored at −70° C. for 1 hour, followed by centrifugation at 4° C. and 14,000 rpm for 10 min to identify an RNA pellet. This was washed with 1 ml of 75% ethanol, dried and resuspended in DEPC-dH₂0 before storage at −70° C. The RNA thus obtained was identified by electrophoresis on 1% agarose gel (0.5% TAE) while the RNA concentration was determined using GeneQuant II (Pharmacia biotech).

3-2. cDNA Synthesis from Genomic RNA

cDNA synthesis was achieved using a cDNA Cycle™ kit (Invitrogen). 400 ng of the RNA was placed into a PCR tube and mixed with DEPC-dH₂O to form a final volume of 11.5 □. It was mixed well with 1 □ of random hexamer and reacted for 10 min in a 65° C. water bath and then for 2 min at room temperature. A mixture of RNase inhibitor 1.0 □, 5×RT buffer 4.0 □, 100 mM dNTPs 1.0 □, 80 mM sodium pyrophosphate 1.0 □ and AMV reverse transcriptase 0.5 □ was added to the tube, which was then tapped slightly, placed for 1 hour in a 42° C. water bath and for 2 min at 95° C. and immediately stored on ice. After the addition of 1.0 □ of 0.5 M EDTA (pH 8.0) and 20 □ of phenol-chloroform, the mixture was vortexed and centrifuged at 4° C. at 14,000 rpm for 15 min. The supernatant thus formed was transferred to a new tube, added with 22 □ of ammonium acetate and 88 □ of 75% ethanol, mixed by vortexing, and stored overnight at −70° C. Following centrifugation at 4° C. and 14,000 rpm for 15 min, the resulting pellet was resuspended in 20 □ of deionized water. cDNA was identified by electrophoresis on 1% agarose gel (0.5% TAE buffer).

3-3. PCR of Nucleoprotein Gene of Rabies Virus Strain ERA

In order to amplify 174 nucleoprotein genes of the Rabies virus strain ERA, which are known to encode T cell epitopes (2), PCR was performed with a set of RVNP-sense primers (5′-ATA CTC GAG GAC GTA GCA CTG GCA GAT G-3′) and RVNP-antisense primers (5′-ATA CTC GAG GTT TGG ACG GGC ATG ACG-3′). First, to a PCR tube was added 1 □ of each of the RVNP-sense primer and the RVNP-antisense primer (2 pmol/□) and 2 □ of the cDNA synthesized at Example 3-2. Finally, 50 □ of a PCR solution containing 5 □ of 10× buffer, 8 □ of dNTP and 1 □ of Taq DNA polymerase (Takara) was prepared. PCR was started by pre-denaturation at 94° C. for 5 min and then with 30 cycles of denaturation at 98° C. for 30 sec, annealing at 54° C. for 30 sec and extension at 72° C. for 30 sec, followed by extension at 72° C. for 5 min. The PCR product was identified by electrophoresis on 1.5% agarose gel (0.5% TAE buffer).

3-4. Construction of RCII/pQE30

The RVNP PCR product was digested with XhoI while the ApoCII/pQE30 vector was treated with XhoI and SalI.

The RVNP PCR digest was ligated overnight to the linearized ApoCII/pQE30 vector in the presence of T4 DNA ligase at 16° C. The resulting recombinant plasmid was transformed into JM109 E. coli and amplified. After preparation from the transformant, the plasmid was treated with SalI and HindIII to identify the insertion of the gene of interest therein in the appropriate direction.

### Example 4

### Construction of pB4RCII Vector

The same B14 fragment inserted into pQE30 as disclosed in Korean Pat. No. 10-0639397 was obtained by treatment with XhoI. Separately, the pQE30 vector carrying the RCII fragment was cut with XhoI and ligated overnight to the B 14 fragment in the presence of T4 DNA ligase at 16° C. to give a recombinant BL4RCII/pQE30(pB4RCII) plasmid. 300-500 ng/□ of pB4RCII was entrusted to Cosmo Co. Ltd. for DNA sequencing. After preparation from E. coli JM109 anchoring the BL4RCII/pQE30 vector, it was treated with SalI to identify the insertion of the gene in the appropriate direction (FIG. 2b). The amino acid sequence of B4RCII is represented by SEQ ID NO.: 9.

### Example 5

### Construction of pB4RB2 Vector

The BX2/pQE30(pB2) vector disclosed in Korean Pat. No. 10-0472841 was digested with SalI and XhoI. The R fragment obtained in Example 3 was ligated overnight to the linearized BX2/pQE30 in the presence of T4 DNA ligase at 16° C. to give a recombinant RBX2/pQE30(pRB2) plasmid.

A B4 fragment, which might be obtained by cutting the pB4RCII of Example 4 with XhoI, was ligated to pTB2, which was also previously treated with XhoI, in the presence of T4 DNA ligase at 16° C. for 15 hours to give a recombinant B4RBX2/pQE30(pB4RB2) plasmid. After preparation from E. coli JM109 anchoring the B4RBX2/pQE30 vector, it was treated with SalI to identify the insertion of the gene in the appropriate direction (FIG. 2c). The amino acid sequence of B4RB2 is represented by SEQ ID NO.: 10.

### Example 6

### Construction of pB4TB2 Vector

The BX2/pQE30(pB2) vector disclosed in Korean Pat. No. 10-0472841 was digested with SalI and XhoI. A T fragment was prepared by digesting the PCR 2.1 vector disclosed in Korean Pat. No. 10-0639397. The T fragment was ligated overnight to the linearized BX2/pQE30 in the presence of T4 DNA ligase at 16° C. to give a recombinant B4TBX2/pQE30(pRB2) plasmid.

A B4 fragment, which was obtained by cutting the pBluescriptII SK 4 with SalI and XhoI, was ligated overnight to pTB2, which was also previously treated with SalI, in the presence of T4 DNA ligase at 16° C. to give a recombinant B4TBX2/pQE30(pB4TB2) plasmid. After preparation from E. coli JM109 anchoring the B4TBX2/pQE30 vector, it was treated with SalI and HindIII to identify the insertion of the gene in the appropriate direction (FIG. 2d). The amino acid sequence of B4TB2 is represented by SEQ ID NO.: 11.

### Example 7

### Expression of Recombinant B4RCII, B4RB2 and B4TB2

M15 for use as a host cell in protein expression was smeared over an LB plate containing ampicillin and kanamycin, and colonies appeared. One of them was cultured overnight in 10 □ of an LB broth containing Amp (50 □/□)□ Kan (50 □/□). 1 □ of the culture was inoculated into 50 □ of a fresh LB broth in order to observe protein induction over time. The culture was incubated at 37° C. for 1.5 hours with shaking to reach an absorbance at 600 nm of 0.4˜0.5, after which IPTG was added at a final concentration of 1 mM, and 1 □ of the culture was sampled at regular intervals of 1 hour during incubation for an additional 5 hours. Prior to IPTG addition, 1 □ of the culture was taken and used as a control. Each culture was centrifuged at 14,000 rpm for 1 min and the pellets thus obtained were resuspended in 30 □ of 2×SDS sample buffer before SDS-PAGE. The proteins were calculated to have a size of 22 kDa for B4RCII, 21 kDa for B4RB2 and 20 kDa for B4TB2. The SDS-PAGE results are given in FIGS. 9(a) to 9(c), showing the expression of the proteins over time.

### Example 8

### Western Blotting for the Recombinant Peptide B4RCII, B4RB2 and B4TB2

The B4RCII, B4RB2 and B4TB2 peptide was identified by size analysis using SDS-PAGE, but in order to further confirm whether the expressed protein is B4RCII, B4RB2 and B4TB2, Western blotting was carried out using two antibodies capable of recognizing B4RCII, B4RB2 and B4TB2. As a control in Western blotting for B4RCII, B4RB2 and B4TB2, E. coli M15 was transformed with the pQE30 vector not containing the B4RCII, B4RB2 and B4TB2 fragment. Samples were collected before IPTG induction and four hours after IPTG induction.

A rabbit anti-PB14 polyclonal antibody was 1:10000 diluted in PBS and used as primary antibodies. As secondary antibodies capable of recognizing the primary antibodies, peroxidase-conjugated goat anti-rabbit IgG was used after being 1:10000 diluted in PBS. A resulting blot was developed using an ECL Plus Western Blotting Kit. The blot was placed in a cassette, and a sheet of Fuji medical X-ray film was placed onto the blot. The blot was exposed to the film for 10 sec and developed. As shown in FIG. 9(d), B4RCII is correctly expressed.

### Example 9

### Identification of Recombinant B4RCII, B4RB2 and B4TB2 in Bacterial Cell

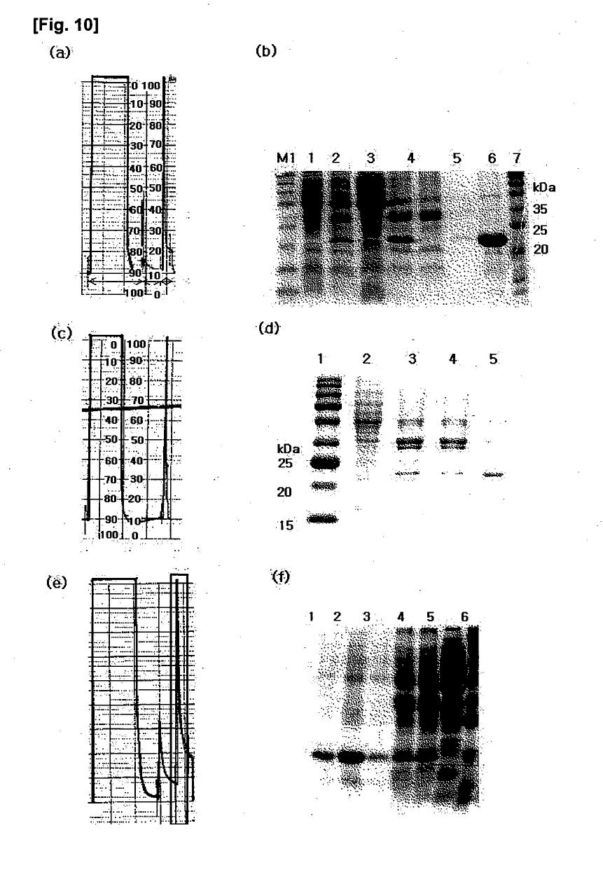

After the cell cultures induced for protein expression as in Example 5 were centrifuged at 4° C. at 9,000 rpm for 30 min, the pellets were frozen for a short time period at −20° C. and thawed on ice. 1 g of each of the pellets was resuspended in 5 □ of a sonication buffer and disrupted by 15 cycles of sonication for 30 sec with an intermission for 1 min per cycle. Centrifugation at 4° C. at 9000 rpm for 30 min gave soluble proteins in the supernatant (crude extract A) and insoluble proteins in the pellet (crude extract B). Each sample was mixed with 2×SDS buffer and boiled at 95° C. for 5 min just before SDS-PAGE (FIG. 10).

### Example 10

### Preparation of Buffers for Purification of Recombinant B4RCII, B4RB2 and B4TB2

Buffers were prepared as follows: 5 mM imidazole, 0.5 M NaCl, 20 mM Tris-C1, pH 7.9 for a sonication buffer; 5 mM imidazole, 0.5 M NaCl, 20 mM Tris-Cl, 8 M Urea, pH 7.9 for binding buffer; 50 mM imidazole, 0.5 M NaCl, 20 mM Tris-Cl, 8 M Urea, pH 7.9 for a washing buffer; 400 mM imidazole, 0.5 M NaCl, 20 mM Tris-Cl, 8 M urea, pH 7.9 for an elution buffer.

### Example 11

### Purification of recombinant B4RCII, B4RB2 and B4TB2

Peptide purification was carried out using Ni-NTA resin(Novagen) for histidine-tagged proteins. This purification is an affinity chromatographic method using the interaction between Ni+ bound to the resin and the histidine hexamer at a N terminal end of a fusion protein. After transformed E. coli cells were pre-cultured in 10 □ of LB medium overnight, the 10-□ culture was inoculated in 500 □ of LB medium and cultured at 37° C. until OD at 600 nm reached 0.4 to 0.5. Then, 1 mM IPTG was added to the medium, and the cells were further cultured for 4 hours. The cells were centrifuged at 9000 rpm for 30 min, and the cell pellet was placed at −20° C. After the frozen cells were thawed on ice, they were resuspended in sonication disruption buffer (5 □/g of wet cells) and sonicated. The cell lysate was then centrifuged at 9000 rpm at 4° C. for 30 min. The pellet was resuspended in a volume of binding buffer equal to that of the supernatant, sonicated three times to remove cell debris, and centrifuged at 9000 rpm at 4° C. for 30 min. The thus obtained supernatant was subjected to affinity chromatography using Ni-NTA resin. A column was 1 cm in diameter and 15 cm in height and was packed with 2 □ of a resin, and all of the steps were carried out at a flow rate of 2 □/min. After the resin was packed into the column, the resin was washed with a three to five column volume of distilled water, and the resin was charged with Ni²⁺ using a five column volume of Ix charge buffer (50 mM NiSO₄) and equilibrated with the binding 4 buffer, thereby generating a Ni-chelate affinity column. After a sample was loaded onto the column twice, the column was washed with the binding buffer until the absorbance at 280 run reached a baseline of 1.0 and then with washing buffer for 10 min. After the column was completely equilibrated, elution buffer was run alone through the column and collected elute proteins. Since the eluted peptide was dissolved in 8 M urea, it was dialyzed in PBS to remove urea. The dialysis was conduced with a slow decrease in urea concentration in order to accurately refold the proteins. Absorbance at 280 nm of the refolded protein fractions in the chromatography was shown in FIGS. 6(a), 6(c) and 6(e). The fractions obtained in various steps were identified by SDS-PAGE, as shown in FIGS. 10(b), 10(d) and 10(f).

### Example 12

### Quantification of Recombinant B4RCII, B4RB2 and B4TB2

Because B4RCII did not aggregate into precipitates, although it was dialyzed with a slow decrease in urea concentration, its amount was determined in such a condition. Protein quantification was conducted using BCA protein assays and UV absorbance. BSA standards for BCA protein assays were prepared by diluting a 2.0 mg/□ BSA stock into 1000, 500, 250, 125, and 62.5 □/□. Samples were reacted with a mixture of 50:1 Reagent A: Reagent B at 37° C. for 30 min and measured for absorbance at 562 nm. Using the standard curve, protein concentrations were determined. As for UV absorbance, protein concentrations were determined by dividing the absorbance at 280 nm by 1.63, which is the E value of B4RCII.

### Example 13

### Immunization of ICR Mice

ICR mice were classified into five groups, including a positive group (diet-induced obesity (DIO)), a negative control (non-DIO, normal group), a B4RCII-immunized group, a B4RB2-immunized group, and a B4TB2-immunized group. 6-week-old ICRmice were injected peritoneally with 100 □ of a solution containing 50 □ of B4RCII, B4RB2 or B4TB2. After injection was repeated three times at regular intervals of two weeks, body weights were monitored and the change was graphed. After the primary boost, a high-fat diet was provided to subject the mice to DIO (diet induced obesity). Blood was sampled from the tail one week after the primary boost and one week, three weeks and five weeks after the secondary boost.

As shown in FIG. 11, the individual mice were similar in body weight, ranging from 22 to 23 g, throughout the groups until the primary injection and boost. From the time of DIO, however, the control (obesity) was found to increase in body weight, whereas the groups injected with B4RCII, B4RB2 or B4TB2 showed only a slight increase in body weight. When they were 14 weeks old (8 weeks after the primary injection), the control and the B4RB2-injected group differed in body weight by approximately 8 g, indicating that the weak immune response induced by the primary injection was boosted by the secondary injection to an extent sufficient to suppress weight gain. After the tertiary injection, the weight gain was measured to remain within the expected deviation range.

### Example 14

### Antibody Titers were Measured Using Serum Samples by Indirect ELISA

100 □ (100 ng) of PB14 was placed into each well of a microtiter plate. The plate was incubated at 4° C. overnight, and incubated in a blocking solution (PBS, 0.5% casein, 0.02% NaN3) at 37° C. for 1 hour. Each well was washed with washing buffer three times. Serum samples collected from Example 10 were 1:1000 to 1:8000 diluted in PBS. 100 □ of each diluted serum sample was added to each well, and incubated at 37° C. for 1 hour. Each well was washed with washing buffer three times and incubated with a 1:1000 dilution of goat anti-mouse IgG as a secondary antibody.

As shown in FIG. 12, the B4RB2- or B4TB2-immunized group increased in antibody titer until 14-week-old, but decreased after that point, while the antibody titer of the B4RCII-immunized group increased until 16-week-old and decreased after that point.

### Example 15

### Evaluation of Serum Lipid Profiles

TG and cholesterol levels were measured as follows. 4 □ of a serum sample were mixed with 200 □ of a development reagent and incubated at 37° C. for 5 min, and absorbance was then measured at 505 nm and 500 nm. To measure HDL levels, a serum sample was mixed with a precipitation reagent at a ratio of 1:1, allowed to stand at room temperature for 10 min, and centrifuged at over 3000 rpm for 10 min. 4 □ of the centrifugal supernatant was mixed with 200 □ of a development reagent and incubated at 37° C. for 5 min, and absorbance was then measured at 555 nm. LDL-cholesterol levels were measured using an EZ LDL cholesterol kit (Sigma) and an LDL calibrator (Randox). According to the protocol supplied by the manufacturer, 4 □ of a serum sample was mixed with 1,150 □ of a reagent contained in the kit, incubated at 37° C. for 5 min, supplemented with 250 □ of the reagent, and incubated again at 37° C. for 5 min. Then, absorbance was measured at 600 nm. Serum levels of each lipid were determined using measured absorbance and a standard curve was obtained using standard solutions.

As for the lipid levels in blood, as shown in FIG. 13, the vaccinated groups were found to be lower in blood total cholesterol (TC), triglyceride (TG), HDL cholesterol and LDL cholesterol lipid levels than was the control (obesity).

### INDUSTRIAL APPLICABILITY

As described hereto, the immunogenic hybrid polypeptides according to the present invention can be applied to mammal animals, such as dogs, cats, cattle, etc., as well as humans. With the ability to induce more uniform and stable immune responses, the hybrid polypeptides are useful in the prevention and treatment of obesity in animals as well as humans.

1. An immunogenic hybrid polypeptide comprising (i) a monomer or a multimer of a peptide having an amino acid sequence selected from the group consisting of SEQ ID NO.: 1, SEQ ID NO.: 2 and SEQ ID NO.: 3; (□) a rabies virus helper T cell epitope, or a hepatitis B virus surface antigen helper T cell epitope; and (□) a C-terminal peptide fragment of mouse apolipoprotein CII, or a monomer or multimer of a peptide having an amino acid sequence selected from the group consisting of SEQ ID NO.: 1, SEQ ID NO.: 2 and SEQ ID NO.: 3, in order in a direction from an N-terminus to a C-terminus thereof.

2. The immunogenic hybrid polypeptide according to claim 1, wherein the multimer of (i) comprises two to eight peptides that each have an amino acid sequence selected from the group consisting of SEQ ID NO.: 1, SEQ ID NO.: 2 and SEQ ID NO.: 3.

3. The immunogenic hybrid polypeptide according to claim 2, wherein the multimer comprises four peptides that each have an amino acid sequence selected from the group consisting of SEQ ID NO.: 1, SEQ ID NO.: 2 and SEQ ID NO.: 3.

4. The immunogenic hybrid polypeptide according to claim 3, wherein the multimer comprises four peptides having an amino acid sequence of SEQ ID NO.: 1.

5. The immunogenic hybrid polypeptide according to claim 4, wherein the multimer has an amino acid sequence of SEQ ID NO.: 5.

6. The immunogenic hybrid polypeptide according to claim 1, wherein the rabies virus helper T cell epitope has an amino acid sequence of SEQ ID NO.: 6.

7. The immunogenic hybrid polypeptide according to claim 1, wherein the hepatitis B virus surface antigen helper T cell epitope has an amino acid sequence of SEQ ID NO.: 7.

8. The immunogenic hybrid polypeptide according to claim 1, wherein the C-terminal peptide fragment of apolipoprotein CII has an amino acid sequence of SEQ ID NO.: 8.

9. The immunogenic hybrid polypeptide according to claim 1, wherein the multimer of (□) comprises two to four peptides that each have an amino acid sequence selected from the group consisting of SEQ ID NO.: 1, SEQ ID NO.: 2 and SEQ ID NO.: 3.

10. The immunogenic hybrid polypeptide according to claim 9, wherein the multimer comprise two peptides that each have an amino acid sequence selected from the group consisting of SEQ ID NO.: 1, SEQ ID NO.: 2 and SEQ ID NO.:

11. The immunogenic hybrid polypeptide according to claim 10, wherein the multimer comprises two peptides having an amino acid sequence of SEQ ID NO.: 1.

12. The immunogenic hybrid polypeptide according to claim 1, comprising (i) a tetramer of a peptide having an amino acid sequence of SEQ ID NO.:1; (□) a rabies virus helper T cell epitope; and (□) a C-terminal peptide fragment of mouse apolipoprotein CII, in order in a direction from an N-terminus to a C-terminus thereof.

13. The immunogenic hybrid polypeptide according to claim 12, having an amino acid sequence of SEQ ID NO.: 9.

14. The immunogenic hybrid polypeptide according to claim 1, comprising (i) a tetramer of a peptide having an amino acid sequence of SEQ ID NO.:1; (□) a rabies virus helper T cell epitope; and (□) a dimer of a peptide having an amino acid sequence of SEQ ID NO.: 1 in order in a direction from the N-terminus to the C-terminus thereof.

15. The immunogenic hybrid polypeptide according to claim 14, having an amino acid sequence of SEQ ID NO.: 10.

16. The immunogenic hybrid polypeptide according to claim 1, comprising (i) a tetramer of a peptide having an amino acid sequence of SEQ ID NO.:1; (□) a hepatitis B virus surface antigen helper T cell epitope; and (□) a dimer of a peptide having an amino acid sequence of SEQ ID NO.: 1, in that order in a direction from the N terminus to the C terminus thereof.

17. The immunogenic hybrid polypeptide according to claim 16, having an amino acid sequence of SEQ ID NO.: 11.

18. A vaccine for the prevention or treatment of obesity, comprising the immunogenic hybrid polypeptide of one of claims 1 to 17 as an active ingredient.

19. A polynucleotide, encoding the immunogenic hybrid polypeptide of one of claims 1 to 17.

20. A recombinant expression vector, comprising the polynucleotide of claim 19.

21. A host cell, transformed with the recombinant expression vector of claim 20.

22. A method for producing the immunogenic hybrid polypeptide of claim 1, comprising culturing the host cell transformed with the recombinant expression vector of claim 20.
